(19) United States
(12) Patent Application Publication
(10) Pub. No.: US 2019/0078982 A1
(43) Pub. Date: Mar. 14, 2019

(54) BIOLOGICAL FLUID TESTING DEVICE

(71) Applicant: Roy G. Whiteside, JR., Belton, TX (US)

(72) Inventor: Roy G. Whiteside, JR., Belton, TX (US)

(21) Appl. No.: 16/006,556

(22) Filed: Jun. 12, 2018

Related U.S. Application Data
(60) Provisional application No. 62/558,838, filed on Sep. 14, 2017.
(60) Provisional application No. 62/558,839, filed on Sep. 14, 2017.

(51) Int. Cl. G01N 1/10; A61B 10/00; B01L 3/00

(52) U.S. Cl. CPC G01N 1/10; G01N 2001/028; B01L 3/508; A61B 10/0051

(57) ABSTRACT

A biological fluid testing and collection device includes a container, a cartridge, and a collection stick. The cartridge is configured to rest within the container and seat itself just above a reservoir at the bottom of the container. The collection stick acquires a liquid sample on a pad and is inserted within the container inside the internal cartridge. The pad is compressed against a screen on the cartridge to release the liquid sample. The liquid sample falls through apertures in the cartridge and collects centrally within the reservoir. The testing strips are located externally around the cartridge and contact the reservoir surface. The testing strips are routed to point centrally in the reservoir and extend beneath the pad. A port in communication with the reservoir may be used to permit the removal of liquid sample within the reservoir without removal of the collection stick.

### CLAIM OF PRIORITY

This application claims the benefit of U.S. Provisional Application No. 62/558,838, filed 14 Sep. 2017, and U.S. Provisional Application No. 62/558,839, filed 14 Sep. 2017. The information contained therein is hereby incorporated by reference.

### BACKGROUND

### 1. Field of the Invention

The present application relates generally to a fluid sampling device, and in particular to a device used to test the presence of selected substances in a biological fluid.

### 2. Description of Related Art

Analytical devices for collecting samples and determining the presence or absence and/or quantifying the amount of various analytes in the samples are known in the market. Assays are usually available for abused drugs, pregnancy and fertility testing, and infectious diseases. Most of these existing analytical devices are typically designed and used to analyze urine samples for testing DOA (Drugs Of Abuse) chemicals. Urine has the traditional problem that the sample is usually collected in private, raising security and handling issues, such as sample tampering.

Therefore, it is preferable in some situations to conduct analysis on a saliva sample rather than on a urine sample. When applicable, saliva collection and analysis has several advantages over urine collection analysis. A saliva specimen can be taken at any convenient time, and can be obtained singly or sequentially. Moreover, saliva sampling may be done at any location and can be easily observed, if required.

Devices for collecting and analyzing saliva samples have been developed and introduced commercially. Such devices are generally divided into two types. The first type adopts a configuration wherein a collecting part is separated from the analyzing part. The second type adopts a configuration similar to a mid-stream urine pregnancy test, where samples are collected on a pad and delivered by capillary action to the testing channel in the same device.

However, the first type of devices requires multiple steps and may raise the issue of sample contamination. The second type of devices, which is a major improvement over the first type, is able to perform the sample collection and assay in one step. However, since the second type of devices has to serve both purposes of sample collecting and sample delivering, it usually requires a soft pad for collecting the sample and a rigid pad for delivering the sample to the testing channel. Further, since the pads cannot be squeezed, it needs a large liquid capacity with minimum retention volume. All these requirements render the designing and selection of padding materials difficult. In addition, since the testing device is attached to the collection pad, it is relatively inconvenient to handle the testing device and very likely to have fluids spilled over the whole device.

A third device is similar to that of the second type of devices, where a single step of collection and assaying is permitted. In this type of device a soft pad is used for collecting the sample and is inserted into a container where it is squeezed to release the sample from the pad. The sample extends outward around an internal reservoir to contact one or more testing strips. A disadvantage of these types of devices is that the distribution of sample is distributed outward around the exterior of the internal container. Sample levels must be large enough to fill the large reservoir. Additionally, tilting of the device could result in not all testing strips contacting the sample. Once the sample is used, it is discarded through the opening in which it entered. With each of the above types of testing devices, once the sample is released into the container, the sample is unable to be safely withdrawn for continued testing if the initial results warrant it. These types of devices require the sample be poured out and captured (unsanitary) or a second sample be taken.

Although great strides have been made with respect to the method of collecting and testing fluid samples, considerable shortcomings remain. A new method and apparatus for the collection and testing of biological fluids is needed that permits operation with minimal sample quantity and ensures each testing strip has access to the sample. Additionally it is desired that the device permit a safe and uncontaminated way of removing the sample from the device if further testing is necessary.

### SUMMARY OF THE INVENTION

It is an object of the present application to provide a biological fluid testing and collection device that provides a container with an inner hollowed cartridge for holding a plurality of testing strips. The cartridge is located in the container so as to situate the testing strips partially at the bottom of the container. The container is concaved along the bottom surface such that the testing strips are directed toward the center of the concave surface when contacting the reservoir surface. Liquid released from a collection stick and swab passes beneath the cartridge and pools within the reservoir. By locating the test strips centrally within the reservoir and by contouring the surface of the reservoir into a concave surface where the center of the surface is the lowest point, less liquid sample is needed to ensure each testing strip is able to absorb the necessary amount of liquid sample.

It is a further object of the present application that the sponge located on the collection stick be increased in size to assist in getting an adequate sample collected. Furthermore it is another object of the present application to increase the length of the collection stick to allow for more compression in the sponge when inserted into the container. Another object of the present application is to permit greater compressive force between the container and the collection stick. The increase in compression of the sponge results in an increase in forces experienced between the collection stick and the container. The contact area is increased therein. The collection stick includes a plurality of fins that extend outward and engage an internal flange in the container. The internal flange can engage one or more fins as the collection stick is inserted into the container.

A further object of the present application is to provide a simplified manner of releasing the liquid sample after collection and testing are complete. A port is optionally located in communication with the reservoir and extends below the reservoir externally on the container

The method of using the assembly is simplified in that a user merely has to gather a liquid sample onto the sponge and insert the collection stick into the container. Within the container is a cartridge with one or more test strips arounds its exterior and that extend below the cartridge. The testing strips make contact with the surface of the reservoir and collect centrally within the reservoir. A user presses the collection stick against the cartridge which allows the fins to engage at least one internal flange so as to secure the collection stick with the sponge in a compressed state. The liquid falls through apertures in the cartridge and collects in the reservoir centrally. The liquid sample is wicked by the testing strips to identify the presence of one or more substances. Ultimately the invention may take many embodiments. In this way, this device overcomes the disadvantages inherent in the prior art.

The more important features of the device have thus been outlined in order that the more detailed description that follows may be better understood and to ensure that the present contribution to the art is appreciated. Additional features of the device will be described hereinafter and will form the subject matter of the claims that follow.

Many objects of the present device will appear from the following description and appended claims, reference being made to the accompanying drawings forming a part of this specification wherein like reference characters designate corresponding parts in the several views.

Before explaining at least one embodiment of the device in detail, it is to be understood that the device is not limited in its application to the details of construction and the arrangements of the components set forth in the following description or illustrated in the drawings. The device is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and devices for carrying out the various purposes of the present device. It is important, therefore, that the claims be regarded as including such equivalent constructions insofar as they do not depart from the spirit and scope of the present device.

### DESCRIPTION OF THE DRAWINGS

The novel features believed characteristic of the application are set forth in the appended claims. However, the application itself, as well as a preferred mode of use, and further objectives and advantages thereof, will best be understood by reference to the following detailed description when read in conjunction with the accompanying drawings, wherein:

FIG. 1 is a perspective view of a biological fluid testing and collection device according to an embodiment of the present application.

FIG. 2 is a side view of the biological fluid testing and collection device of FIG. 1.

FIG. 3 is a section view of the biological fluid testing and collection device of FIG. 1.

FIG. 4 is an exploded view of the biological fluid testing and collection device of FIG. 1.

FIG. 5 is a side view of a collection stick used in the biological fluid testing and collection device of FIG. 1.

FIGS. 6 and 7 are perspective views of a container used in the biological fluid testing and collection device of FIG. 1.

FIG. 8 is a side view of an internal cartridge used in the biological fluid testing and collection device of FIG. 1.

FIG. 9 is lower perspective view of the internal cartridge of FIG. 8.

FIGS. 10-12 are side views of the biological fluid testing and collection device of FIG. 1 illustrating the steps of use.

While the device and method of the present application is susceptible to various modifications and alternative forms, specific embodiments thereof have been shown by way of example in the drawings and are herein described in detail. It should be understood, however, that the description herein of specific embodiments is not intended to limit the application to the particular embodiment disclosed, but on the contrary, the intention is to cover all modifications, equivalents, and alternatives falling within the spirit and scope of the process of the present application as defined by the appended claims.

### DETAILED DESCRIPTION OF THE PREFERRED EMBODIMENT

Illustrative embodiments of the preferred embodiment are described below. In the interest of clarity, not all features of an actual implementation are described in this specification. It will of course be appreciated that in the development of any such actual embodiment, numerous implementation-specific decisions must be made to achieve the developer's specific goals, such as compliance with device-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms to describe a spatial relationship between various components or to describe the spatial orientation of aspects of such components should be understood to describe a relative relationship between the components or a spatial orientation of aspects of such components, respectively, as the device described herein may be oriented in any desired direction.

The device and method in accordance with the present application overcomes one or more of the above-discussed problems commonly associated with elevated platforms discussed previously. In particular, the biological fluid testing and collection device is configured to provide an increased pad to collect a liquid sample, an increases length of collection stick to provide more compression of the collection pad (i.e. sponge), and an interface between a collection stick and the container that yields greater retention forces. The device is configured to centrally locate the collection of liquid sample within a reservoir and locate ends of the testing strips centrally within the reservoir so as to increase activation of the testing strips, minimize the need of liquid sample needed and so forth. The device is also optionally configured to provide for the removal or discharge of liquid sample while the device is closed with the collection stick seated on the container. These and other unique features of the device are discussed below and illustrated in the accompanying drawings.

The device and method will be understood, both as to its structure and operation, from the accompanying drawings, taken in conjunction with the accompanying description. Several embodiments of the device may be presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless otherwise described.

The device and method of the present application is illustrated in the associated drawings. The device includes a container, a collection stick, and an internal cartridge. The cartridge is configured to rest within the container and seat itself just above a reservoir at the bottom of the container. The collection stick acquires a liquid sample and is inserted within the container inside the hollowed internal cartridge and is compressed against a lower inner surface of the cartridge such that the pad releases the liquid sample upon compression and the seating of the collection stick in the container. The liquid sample falls through apertures in the cartridge and collects centrally within the reservoir. The testing strips are located externally around the cartridge and contact the reservoir surface and are routed to point centrally in the reservoir. Additional features and functions of the device are illustrated and discussed below.

Referring now to the Figures wherein like reference characters identify corresponding or similar elements in form and function throughout the several views. The following Figures describe the assembly of the present application and its associated features. With reference now to the Figures, an embodiment of the biological fluid testing and collection device and method of use are herein described. It should be noted that the articles “a”, “an”, and “the”, as used in this specification, include plural referents unless the content clearly dictates otherwise.

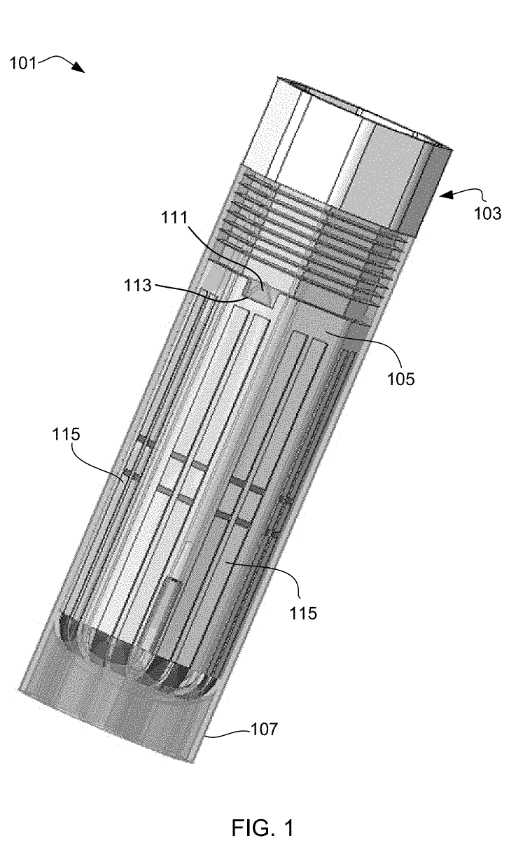

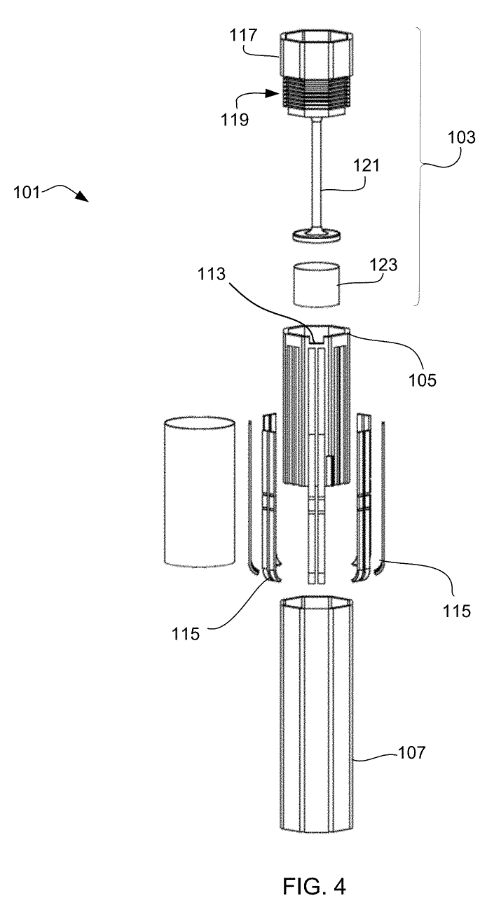

Referring now to FIGS. 1-4 in the drawings, assorted views of a biological fluid testing and collection device 101 are illustrated. Device 101 is configured to collect a fluid sample from a patient via a swab pad and selectively discharge the sample when the swap pad contacts a screen located within a cartridge. The sample is funneled centrally within a container for reception by a plurality of testing strips. The testing strips are partially angled relative to the axis of the container so as to extend toward the center (and lowest point) of the reservoir. This angling is to assist in minimizing sample volume required to obtain a successful testing and to minimize the effects of tilt on the container. The device is also optionally configured to include a port at the base of the reservoir for the withdrawal of sample from the container.

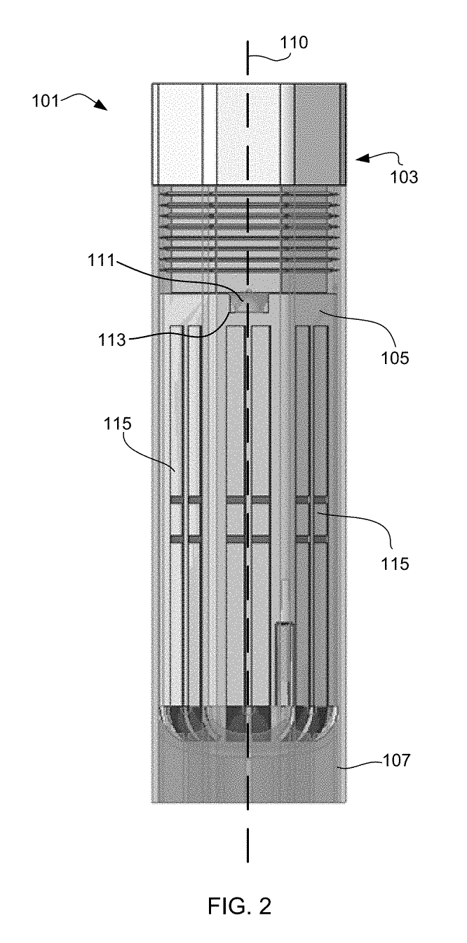

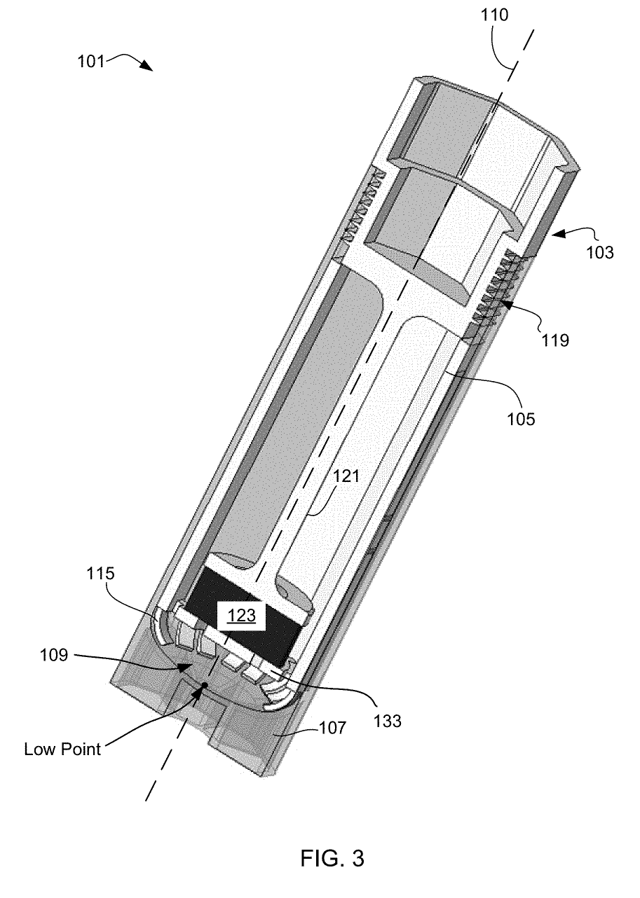

FIG. 1 illustrates a perspective view of device 101. In FIG. 2 is shown a side view of device 101. To gain more insight into device 101, FIG. 3 is a section view of device 101 as seen in FIG. 1. Device 101 includes a collection stick 103, an internal cartridge 105, and a container 107. Cartridge 105 is configured to translate within and be seated internally within container 107. Collection stick 103 is configured to translate within internal cartridge 105 and container 107, and contact a screen. Device 101 is configured to selectively withdraw a sample from collection stick 103 when located in internal cartridge 105. Device 101 is configured to centrally locate the released sample within a reservoir 109, pooled centrally.

As seen in FIGS. 1-3, cartridge 105 is internally seated within container 107. Both container 107 and cartridge 105 share the same longitudinal shape wherein container 107 is sized larger that cartridge 105. As seen in the Figures, container 107 has an octagonal shape with 8 sides of equal size. It is understood that other shapes are possible and as such device 101 is not limited to the longitudinal shape shown herein. The interaction or engagement between cartridge 105 and container 107 is such that cartridge 105 is restricted from rotational movement about an axis 110. Axis 110 is concentric with the axis of container 107. As sides of container 107 are not fully circular, rotational movement between them is not permitted. Container 107 includes an internal protrusion 111 which extends inward into the central void of container 107 along one of its sides. Internal cartridge 105 includes a detent 113 along an upper surface. As cartridge 105 is translated down within container 107, protrusion 111 slides along the side and engages detent 113. Cartridge 105 is seated between reservoir 109 and protrusion 111.

As seen in FIGS. 1-3, collection stick 103 is shown fully inserted into container 107. In particular with FIG. 3, the section view provides more detail. Cartridge 105 further includes testing strips 115 that extend down into reservoir 109. Strips 115 make contact with the lower surface of reservoir 109 and bend or fold inward toward axis 110. Reservoir 109 is configured to have a concave shape such that reservoir 109 is deepest at the center along axis 110. The shape forces the liquid sample to collect centrally. The shape also forces the ends of the testing strips 115 into the center so as to increase the opportunity to obtain the liquid sample. By providing for the central collection of liquid sample and the central locating of strips 115, the odds of collection for each strip 115 is greatly increased.

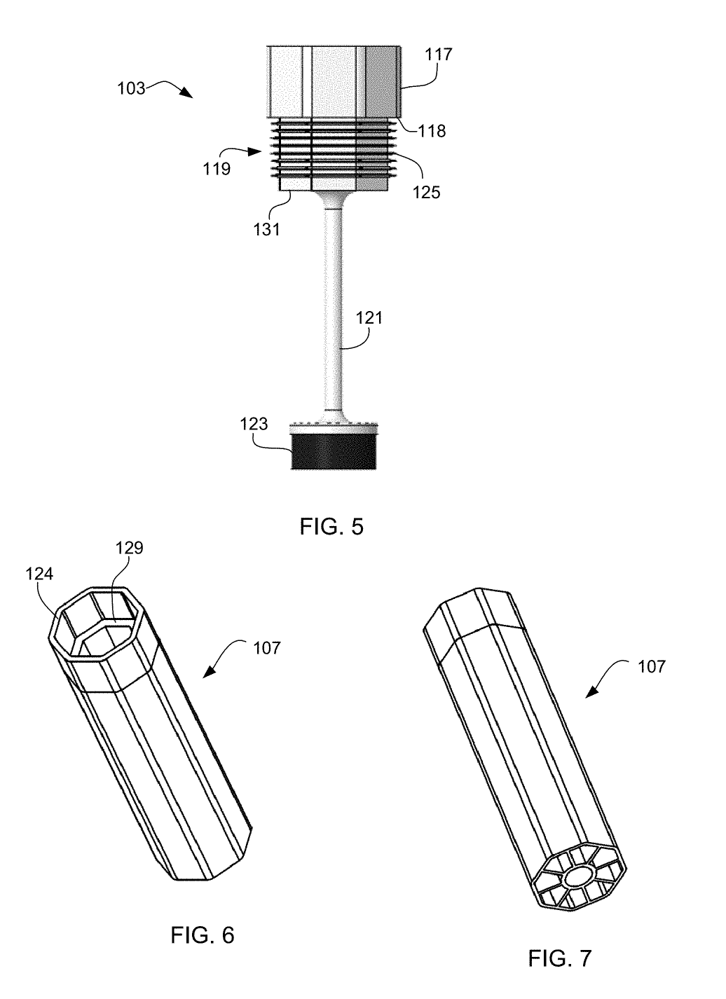

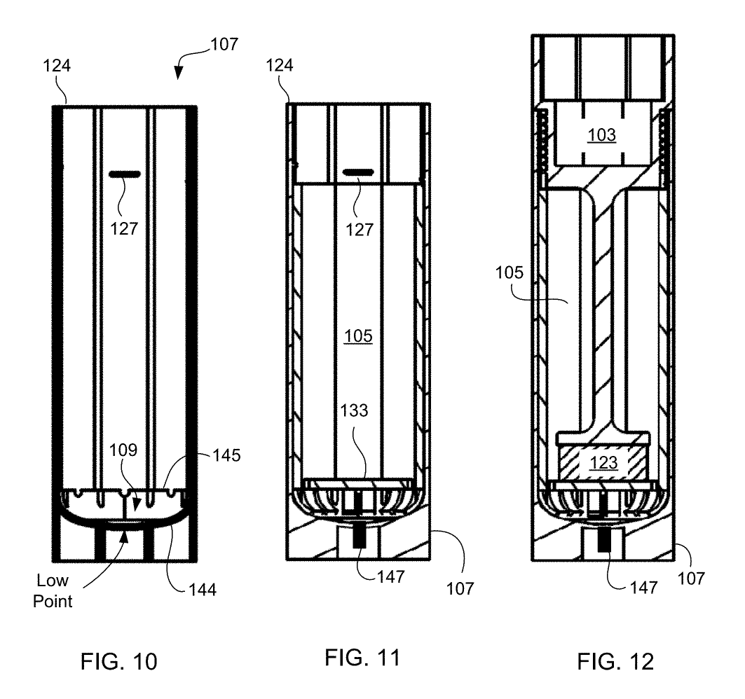

Referring now also to FIG. 5 in the drawings, a side view of collection stick 103 is illustrated. Stick 103 is configured to facilitate the collection of a liquid sample from a patient and permit for the subsequent discharge of the liquid sample into a collection device. Stick 103 includes an upper portion 117, an engagement section 119, a rod 121, and a swab pad 123. Upper portion 117 remains exposed when stick 103 is fully seated in container 107 and acts as a handle for grasping by a user. Upper portion 117 includes a rim surface 118 which contacts an upper surface 124 of container 107 (see FIG. 6). Engagement section 119 is configured to include a plurality of fins 125 that extend outward from its body beneath surface 118. Fins 125 extend radially in a planar fashion around the circumference of section 119. It is understood that fins 125 are not necessarily around the entire perimeter of section 119 but may actually cover one or more surfaces to any degree about its perimeter. Each fin 125 or set of fins 125 are planar to its neighboring fin 125. In this manner, the fins do not in fact create a threaded relationship with container 107. Container 107 includes an internal flange 127 (see FIGS. 10-11) which extends inward toward the central void of container 107 similar to that of protrusion 111. Fins 125 are configured to flex and pass by flange 127 as stick 103 is inserted and seated. The location or depth from surface 124 that flange 127 is located has an influence on the number of fins 125 that engage with and/or pass flange 127. Multiple flanges 127 may be used. It is understood that their depth below surface 124 may differ for each. The stiffness of fins 125 is sufficient to overcome the compressive forces needed to compress swab pad 123.

Referring now also to FIGS. 6 and 7 in the drawings, views of container 107 are illustrated. Container 107 includes an internal lip 129. Lip 129 is configured to contact a bottom surface 131 of section 119. The distance between surface 124 and lip 129 is relatively equal to that of the distance between surface 118 and surface 121 on stick 103. These distances have been lengthened to permit more room to engage each other and to increase the forces exerted upon stick 103 when swab pad 123 is compressed. As seen in FIG. 5, rod 121 extends down from section 119 along its central axis and is used to locate swab pad 123 at a distance from section 119. When inserted in container 107, passes down through cartridge 105 and presses swab pad 123 against a screen 133. Rod 121 is lengthened to get a greater amount of compression in swab 123. When fully seated, stick 103 compresses swab 123 fully. Full compression of swab 123 ensures that as much of the liquid sample as possible is put to use.

Swab 123 is configured to absorb a liquid sample from a patient and facilitate its transfer to a collection device. This is taken most likely through the mouth where saliva is absorbed into the sponge. Other types of bodily fluids are conceived as able to be captured and a saliva sample is not meant to be limiting. An example of a material for swab 123 is a sponge material, one that is absorbent upon contact and able to discharge collected fluid upon compression. The size of swab 123 is also increased (i.e. diameter). An exemplary diameter size can be anything up to 19.05 mm.

It is understood that in some embodiments, lip 129 may not be included. In such an embodiment, surface 131 may in fact may contact with cartridge 105 instead. This is seen through FIGS. 1-3, whereas the lip is shown for exemplary purposes in FIG. 6. FIG. 7 is a lower perspective view of container 107.

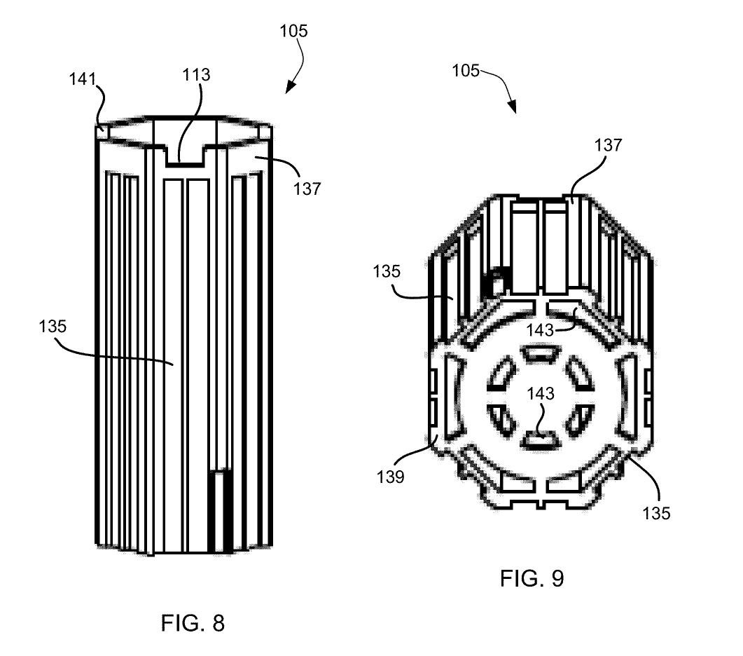

Referring now also to FIGS. 8 and 9 in the drawings, a side view and a lower perspective view of cartridge 105 are illustrated, respectively. Cartridge 105 is configured to locate one or more testing strips 115 within container 107 such that the testing strips 115 extend down into reservoir 109 to await the liquid sample. Cartridge 105 includes one or more vertical grooves 135 along its outer surface 137. Grooves 135 extend the length of cartridge 105 such that they extend through bottom surface 139. Cartridge 105 also has an upper surface 141. Grooves 135 do not pass through surface 141. Strips 115 are located within grooves 135. The size of the strips 115 are such that they extend downward beyond surface 139. When inserted into container 107, strips 115 contact the surface of reservoir 109 and are folded inward so as to be angled toward the center of the reservoir 109. This acts to centrally locate the tips of the strips 115 in a shared adjacent area.

Cartridge 105 also includes screen 133. Screen 133 is located at the base of cartridge 105 and is configured to include one or more apertures 143 to permit the liquid sample to fall into reservoir 109. Ideally, screen 133 is flush with surface 139 but is not limited therein. The liquid sample is allowed to drop directly on the testing strips 115 prior to contacting the reservoir surfaces. Alternatively, the collection of liquid sample in reservoir 109 can rise to contact each testing strip.

Referring now also to FIG. 10 in the drawings, a side section view of container 107 is illustrated. In this view, lip 129 is not shown. Also not seen is protrusion 111. In FIG. 10 it becomes simpler to describe reservoir 109. Reservoir 109 is the space or volume that is located beneath cartridge 105. This is defined by the base of container 107 that includes a concave inner surface 144 wherein the lowest point is the center point on axis 110. As noted previously, cartridge 105 is seated in container 107 between protrusion 111 and a top rim 145 extending inward from the inner surface of container 107. Surface 139 makes contact with rim 145. It is understood that rim 145 may be the inflection point wherein the vertical walls of container 107 proceed to take a radial arc that forms the surface of the reservoir 109. In this case, the rim 145 may not actually be an inward protruding surface apart from the normal curved nature of the beginnings of reservoir 109; wherein cartridge 105 contacts the inner surface of container 107.

As noted previously, the internal walls 144 of reservoir 109 are concaved and angled centrally to a low point so as to collect the sample fluid in the middle of container 107. This centrally locates the sample along with the test strips at the center of reservoir 109. Additionally, the tilting of container 107 minimally affects the access of each test strip with the sample. The walls of the container are at least partially transparent to allow an operator to visually see the test results without needed to open container 107.

Referring now also to FIGS. 11 and 12 in the drawings, additional side section views are provided of device 101 to illustrate the steps of operation. As seen in FIG. 9, the container 107 was obtained. In FIG. 11, cartridge 105 is selected and inserted. Cartridge 105 includes one or more styles of strips 115. Strips 115 are located beneath screen 133. The ends of strips 115 that are below surface 139 curve toward the center point of reservoir 109 upon contact with the inner surface of reservoir 109. In FIG. 12, stick 103 is shown engaged and seated in container 107.

As seen in FIGS. 11 and 12 in particular, device 101 is shown to include an optional port 147. Port 147 is located at the bottom of container 107 and is configured to permit the selective draining of liquid sample from the reservoir 109. The port 147 is in fluid communication with reservoir 109. Port 147 may include a needle with a rubber/latex coating, wherein the coating is punctured when the needle is pressed against a collection vial. The needle is safely recessed within the external recesses of the base of the container 107 to avoid inadvertent injury or puncture. The liquid sample may be suctioned out through vacuum or be drained out through port 147. Other collection methods may be used with the port 147. The port 147 facilitates safe removal of the sample while stick 103 is seated so as to also avoid contamination. This is ideal where the test strips identify a result that may require additional tests or the presence of drugs or disease. The sample may then be safely removed and used in subsequent tests or discarded safely without fear of exposure to people.

The particular embodiments disclosed above are illustrative only, as the application may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. It is apparent that an application with significant advantages has been described and illustrated. Although the present application is shown in a limited number of forms, it is not limited to just these forms, but is amenable to various changes and modifications without departing from the spirit thereof.

What is claimed is:

1. A biological fluid testing and collection device, comprising:
a container having an opening and defining a central volume;
a cartridge configured to translate within the central volume and be seated within the container, the cartridge having one or more grooves that pass through a bottom surface of the cartridge, the cartridge including one or more testing strips located within the one or more grooves, the testing strips extend lower than the bottom surface;
a collection stick configured to acquire a liquid sample, the collection stick having a swab pad to absorb a liquid and subsequently release the liquid when compressed, the collection stick being partially inserted into the container and passing the swab pad within the cartridge, the swab pad being compressed against a screen coupled to the cartridge;
wherein the container has an internal curved bottom surface beneath the cartridge that defines a reservoir, the reservoir having a centrally located low point to collect the liquid sample discharged from the swab pad, the testing strips being partially located beneath the screen.

2. The device of claim 1, wherein the testing strips contact the curved surface of the reservoir and are angled toward the low point.

3. The device of claim 1, wherein the discharged liquid sample falls through an aperture in the screen directly onto the testing strip.

4. The device of claim 1, wherein the discharged liquid sample falls through an aperture in the screen and collects in the center of the reservoir.

5. The device of claim 1, wherein the low point is located along an axis of the container, the testing strips being directed to the axis of the container.

6. The device of claim 1, wherein the container includes a lip adjacent the opening of the container, the collection stick configured to contact the lip.

7. The device of claim 1, wherein the collection stick is configured to contact an upper surface of the cartridge.

8. The device of claim 1, wherein the collection stick includes an engagement section which includes one or more fins.

9. The device of claim 1, wherein the one or more fins extend around the perimeter of the engagement section.

10. The device of claim 1, wherein the one or more fins are stacked in a planar manner such that each is parallel to each other.

11. The device of claim 1, further comprising:
an internal flange extending inwardly into the central volume from a surface of the container, the internal flange configured to engage with the collection stick so as to restrict removal thereof.

12. The device of claim 1, wherein the container includes a protrusion extending inwardly into the central volume, the protrusion configured to engage the cartridge.

13. The device of claim 1, wherein the cartridge includes a detent along an upper surface, the detent configured to accept the protrusion.

14. The device of claim 1, wherein the container is configured to permit the discharge of the liquid sample without removal of the collection stick by passing the liquid sample through a port in the container.

15. The device of claim 1, further comprising:
a port in fluid communication with the low point in the reservoir, the port configured to permit the sanitary discharge of the liquid sample.

16. The device of claim 1, wherein the liquid sample can be reused for additional tests.

17. The device of claim 1, wherein the central collection of liquid sample and the angling of the testing strips toward the low point of the reservoir minimize the effects of the container being out of level.

18. The device of claim 1, wherein the swab pad has a diameter up to 19.05 mm.
